SEQUENCE LISTING (84 sequences)

SEQ ID NO: 1 - protein, 11 aa, Homo sapiens
RAPGEDPMDYK

SEQ ID NO: 2 - protein, 22 aa, Homo sapiens
REPPVPPATADPFLLAESDEAK

SEQ ID NO: 3 - protein, 26 aa, Homo sapiens
KMCDPGMTAFEPEALGNLVEGLDFHR

SEQ ID NO: 4 - protein, 26 aa, Homo sapiens
KICDPGLTSFEPEALGNLVEGMDFHR

SEQ ID NO: 5 - protein, 26 aa, Homo sapiens
KICDPGLTAFEPEALGNLVEGMDFHR

SEQ ID NO: 6 - protein, 33 aa, Homo sapiens
KLVEVLDDPNEDHLYMVFELVNQGPVMEVPTLK

SEQ ID NO: 7 - protein, 22 aa, Homo sapiens
KVESLAMFLGELSLIDADPYLK

SEQ ID NO: 8 - protein, 16 aa, Homo sapiens
KYFDSCNGDLDPEIVK

SEQ ID NO: 9 - protein, 20 aa, Homo sapiens
KVLPNDPVFLEPHEEMTLCK

SEQ ID NO: 10 - protein, 20 aa, Homo sapiens
KDLAHTPSQLEGLDPATEAR

SEQ ID NO: 11 - protein, 10 aa, Homo sapiens
KLLVLDPAQR

SEQ ID NO: 12 - protein, 23 aa, Homo sapiens
RIDSDDALNHDFFWSDPMPSDLK

SEQ ID NO: 13 - protein, 17 aa, Homo sapiens
READPALNVETEIEILK

SEQ ID NO: 14 - protein, 20 aa, Homo sapiens
RSEIQVLEHLNTTDPNSTFR

SEQ ID NO: 15 - protein, 9 aa, Homo sapiens
KMLEYDPAK

SEQ ID NO: 16 - protein, 9 aa, Homo sapiens
KEYIDPETK

SEQ ID NO: 17 - protein, 21 aa, Homo sapiens
RLEANMLEPPFCPDPHAVYCK

SEQ ID NO: 18 - protein, 11 aa, Homo sapiens
RLLDPPDPLGK

SEQ ID NO: 19 - protein, 15 aa, Homo sapiens
KPQNLLVDPDTAVLK

SEQ ID NO: 20 - protein, 20 aa, Homo sapiens
RVADPDHDHTGFLTEYVATR

SEQ ID NO: 21 - protein, 30 aa, Homo sapiens
RIEVEQALAHPYLEQYYDPSDEPIAEAPFK

SEQ ID NO: 22 - protein, 11 aa, Homo sapiens
KPVVDPSRITR

SEQ ID NO: 23 - protein, 28 aa, Homo sapiens
RAQSLGLLGDEHWATDPDMYLQSPQSER

SEQ ID NO: 24 - protein, 20 aa, Homo sapiens
RTDPHGLYLSCNGGTPAGHK

SEQ ID NO: 25 - protein, 22 aa, Homo sapiens
RTWHAQISTSNLYLPQDPTVAK

SEQ ID NO: 26 - protein, 17 aa, Homo sapiens
KNLIPMDPNGLSDPYVK

SEQ ID NO: 27 - protein, 30 aa, Homo sapiens
KEPWPLMELVPLDPENGQTSGEEAGLQPSK

SEQ ID NO: 28 - protein, 11 aa, Homo sapiens
KADPSHFELLK

SEQ ID NO: 29 - protein, 10 aa, Homo sapiens
KMLHVDPHQR

SEQ ID NO: 30 - protein, 18 aa, Homo sapiens
RHFDPEFTEEPVPNSIGK

SEQ ID NO: 31 - protein, 10 aa, Homo sapiens
RNSDPNDPNR

SEQ ID NO: 32 - protein, 9 aa, Homo sapiens
KALDPMMER

SEQ ID NO: 33 - protein, 10 aa, Homo sapiens
KVMIHPDPER

SEQ ID NO: 34 - protein, 11 aa, Homo sapiens
KGNDPLGEWER

SEQ ID NO: 35 - protein, 10 aa, Homo sapiens
KICMNEDPAK

SEQ ID NO: 36 - protein, 21 aa, Homo sapiens
KQNADPSLISWDESGVDFYSK

SEQ ID NO: 37 - protein, 39 aa, Homo sapiens
RGLSGSDPTLNYNSLSPLEGPPNHSTSQGPQPGSDPWPK

SEQ ID NO: 38 - protein, 19 aa, Homo sapiens
KLSFQNVDPLGENIRVIFK

SEQ ID NO: 39 - protein, 17 aa, Homo sapiens
RGLQLLQDGNDPDPYVK

SEQ ID NO: 40 - protein, 10 aa, Homo sapiens
KIYLLPDPQK

SEQ ID NO: 41 - protein, 13 aa, Homo sapiens
RTDSASADPGNLK

SEQ ID NO: 42 - protein, 11 aa, Homo sapiens
KLEGRDVDPNR

SEQ ID NO: 43 - protein, 21 aa, Homo sapiens
KPYVDLQAYEDPAQGALDFTR

SEQ ID NO: 44 - protein, 24 aa, Homo sapiens
RELDPAWLMVDTVIGEGEFGEVYR

SEQ ID NO: 45 - protein, 18 aa, Homo sapiens
RGPELEMLAGLPTSDPGR

SEQ ID NO: 46 - protein, 9 aa, Homo sapiens
KHTTDPTEK

SEQ ID NO: 47 - protein, 9 aa, Homo sapiens
KQVDPGLPK

SEQ ID NO: 48 - protein, 19 aa, Homo sapiens
KCFCEPEDIQDPLCDELCR

SEQ ID NO: 49 - protein, 13 aa, Homo sapiens
KAVNGAENDPFVR

SEQ ID NO: 50 - protein, 18 aa, Homo sapiens
RFDVHQLANDPYLLPHMR

SEQ ID NO: 51 - protein, 15 aa, Homo sapiens
KPQNLLVDPDTAVLK

SEQ ID NO: 52 - protein, 9 aa, Homo sapiens
KEYIDPETK

SEQ ID NO: 53 - protein, 31 aa, Homo sapiens
KFFVDGQWTHDPSEPIVTSQLGTVNNIIQVK

SEQ ID NO: 54 - protein, 29 aa, Homo sapiens
KDTGISCDPALLPEPNHVMLNHLYALSIK

SEQ ID NO: 55 - protein, 22 aa, Homo sapiens
KHYLNCSHCEVDECHMDPESHK

SEQ ID NO: 56 - protein, 29 aa, Homo sapiens
RIEYCTQDPDVINTALPIEYGPLVEEEEK

SEQ ID NO: 57 - protein, 17 aa, Homo sapiens
RTLDPNSAFLHTLDQQK

SEQ ID NO: 58 - protein, 15 aa, Homo sapiens
KMIEVDPEAPTEEEK

SEQ ID NO: 59 - protein, 36 aa, Homo sapiens
RGTQLFEDNYALAVLDNGDPLNNTTPVTGASPGGLR

SEQ ID NO: 60 - protein, 16 aa, Homo sapiens
KGLQSLPTHDPSPLQR

SEQ ID NO: 61 - protein, 15 aa, Homo sapiens
KLSFQNVDPLGENIR

SEQ ID NO: 62 - protein, 15 aa, Artificial Sequence
  Description of Artificial Sequence Synthetic peptide
KLSNQNVDPLGEFIR

SEQ ID NO: 63 - protein, 15 aa, Artificial Sequence
  Description of Artificial Sequence Synthetic peptide
KLSFNNVDPLGEQIR

SEQ ID NO: 64 - protein, 21 aa, Homo sapiens
KPYVDLQAYEDPAQGALDFTR

SEQ ID NO: 65 - protein, 21 aa, Artificial Sequence
  Description of Artificial Sequence Synthetic peptide
KPYFDLQAYEDPAQGALDVTR

SEQ ID NO: 66 - protein, 21 aa, Artificial Sequence
  Description of Artificial Sequence Synthetic peptide
KPYVDLQGYEDPAQAALDFTR

SEQ ID NO: 67 - protein, 16 aa, Homo sapiens
KGLQSLPTHDPSPLQR

SEQ ID NO: 68 - protein, 16 aa, Artificial Sequence
  Description of Artificial Sequence Synthetic peptide
KGLQSLPTDPHSPLQR

SEQ ID NO: 69 - protein, 16 aa, Artificial Sequence
  Description of Artificial Sequence Synthetic peptide
KGLQSLPSHDPTPLQR

SEQ ID NO: 70 - protein, 7 aa, Homo sapiens
PLGENIR

SEQ ID NO: 71 - protein, 7 aa, Artificial Sequence
  Description of Artificial Sequence Synthetic peptide
PLGEFIR

SEQ ID NO: 72 - protein, 7 aa, Artificial Sequence
  Description of Artificial Sequence Synthetic peptide
PLGEQIR

SEQ ID NO: 73 - protein, 10 aa, Homo sapiens
PAQGALDFTR

SEQ ID NO: 74 - protein, 10 aa, Artificial Sequence
  Description of Artificial Sequence Synthetic peptide
PAQGALDVTR

SEQ ID NO: 75 - protein, 10 aa, Artificial Sequence
  Description of Artificial Sequence Synthetic peptide
PAQAALDFTR

SEQ ID NO: 76 - protein, 6 aa, Homo sapiens
PSPLQR

SEQ ID NO: 77 - protein, 7 aa, Artificial Sequence
  Description of Artificial Sequence Synthetic peptide
PHSPLQR

SEQ ID NO: 78 - protein, 6 aa, Artificial Sequence
  Description of Artificial Sequence Synthetic peptide
PTPLQR

SEQ ID NO: 79 - protein, 15 aa, Homo sapiens
GLQSLPTHDPSPLQR

SEQ ID NO: 80 - protein, 15 aa, Artificial Sequence
  Description of Artificial Sequence Synthetic peptide
GLQSLPTDPHSPLQR

SEQ ID NO: 81 - protein, 15 aa, Artificial Sequence
  Description of Artificial Sequence Synthetic peptide
GLQSLPSHDPTPLQR

SEQ ID NO: 82 - protein, 14 aa, Artificial Sequence
  Description of Artificial Sequence Synthetic peptide
GGGGGGDPGGGGGG

SEQ ID NO: 83 - protein, 7 aa, Artificial Sequence
  Description of Artificial Sequence Synthetic peptide
GGGGGGD

SEQ ID NO: 84 - protein, 7 aa, Artificial Sequence
  Description of Artificial Sequence Synthetic peptide
PGGGGGG

(19) United States
(12) Patent Application Publication
(10) Pub. No.: US 2009/0253156 A1
(43) Pub. Date: Oct. 8, 2009

(54) MASS SPECTROMETRY METHODS FOR MULTIPLEXED QUANTIFICATION OF PROTEIN KINASES AND PHOSPHATASES

(75) Inventors: Wayne F. Patton, Newton, MA (US); Bing Xie, Milford, MA (US)

Correspondence Address: WILMER, HALE, PERKIN & ELMER, LLP, 60 STATE STREET, BOSTON, MA, 02109, US

(73) Assignee: PerkinElmer LAS, Inc.

(21) Appl. No.: 11/744,705

(22) Filed: May 4, 2007

Related U.S. Application Data
(60) Provisional application No. 60/798,436, filed on May 5, 2006.

(51) Int. Cl. C12Q 1/48; C12Q 1/37

(52) U.S. Cl. 435/15; 435/23

(57) ABSTRACT

The inventions relates to methods and kits for capture and/or analysis of kinases and/or phosphatases in one or more samples. In some embodiments, a kinase inhibitor, e.g. staurosporine or its derivative, is used to capture kinases from a sample. In some embodiments, a phosphatase inhibitor, e.g. microcystin or its derivative, is used to capture phosphatases from a sample. Methods for quantitative analysis of captured kinases and/or proteases are also provided. In some embodiments, quantitative analysis is accomplished using mass spectrometry. In addition, the invention provides kits related to same.

### 1. REFERENCE TO RELATED APPLICATIONS

This application claims the benefit under 35 U.S.C. §119(e) of U.S. Provisional Patent Application No. 60/798,436, filed on May 5, 2006, entitled Mass Spectrometry Methods for Multiplexed Quantification of Protein Kinases and Phosphatases, which is incorporated herein by reference in its entirety.

### 2. BACKGROUND

Precise targeting of specific aspects of kinase cascades is now known to provide previously unattainable breakthroughs for disease therapies. The importance of the protein kinase family is underscored by the numerous disease states that arise due to disregulation of kinase activity. Aberrant cell signaling by many of these protein and lipid kinases can lead to diseases, such as cancer, Alzheimer's disease, and type II diabetes.

Several protein serine/threonine and tyrosine kinases are known to be activated in cancer cells and to drive tumour growth and progression. Blocking protein kinase activity therefore represents a rational approach to cancer therapy. For example, Iressa® (Gefitinib) belongs to a group of anticancer drugs called epidermal growth factor receptor-tyrosine kinase inhibitors (EGFR-TKI). Iressa® blocks several tyrosine kinases, including one associated with Epidermal Growth Factor Receptor (EGFR). EGFR is found on the cell surface of many normal cells and cancer cells. Iressa® works by binding to the tyrosine kinase of the EGFR to directly block growth signals turned on by triggers outside or inside the cell. The drug is used as a single agent treatment for non-small cell lung cancer (NSCLC), being approved for use in patients whose cancer had gotten worse despite treatment with platinum-based and docetaxel chemotherapy. Recent studies indicate some patients have developed mutations that cause resistance to Iressa®. For example, it has been found that the T790M mutation leads to high-level functional resistance to Iressa®. In patients with tumors bearing Iressa®-sensitive mutations (eg. L858R, L861Q), resistant subclones containing the T790M mutation emerge in the presence of the drug. The amino acid substitution L858R is one of several heterozygous mutations that have been identified in Non-Small-Cell Lung Cancer (NSCLC) patients who have clinical responses to the EGFR inhibitor Iressa®. There is some evidence that these mutations result in elevated activity and enhanced sensitivity to Iressa®. Advanced tools such as high-throughput screening, single nucleotide polymorphism (SNP) arrays, exon resequencing, and structural analysis are now being used to help better understand the targets, the mutations, and which patients will most likely respond to more potent, second-generation compounds. Kinase targets are expected to be broadened in the future to inflammatory, autoimmune, central nervous system, and cardiovascular diseases.

A plethora of other protein kinases are now known to be central to a wide variety of diseases. Platelet-derived growth factor receptor alpha (PDGFRα) is a tyrosine kinase receptor involved in regulating essential cell processes such as cell proliferation, motility and survival. The V561D substitution is an activating mutation found in some patients with gastrointestinal stromal tumors. Abl is a non-receptor tyrosine kinase. Chromosomal translocations involving Abl and the breakpoint cluster region on chromosme 22 produce the bcr-abl fusion protein, resulting in a constitutively active Abl, thought to be critical in the pathogenesis of chronic myelogenous leukemia (CML).

Akt/Protein kinase B (PKB) is a serine/threonine kinase known to be a major effector of the PI 3 kinase pathway in response to growth factors or insulin. Mis-regulation of Akt/PKB's activity has been shown to contribute to various human diseases including atherosclerosis and diabetes mellitus. As key regulators of cell division, the Aurora family of serine/threonine kinases, including Aurora A, B and C, have been identified to have direct but distinct roles in mitosis. Over-expression of these three isoforms have been linked to a diverse range of human tumor types, including leukemia, colorectal, breast, prostate, pancreatic, melanoma and cervical cancers. The Axl family of receptor tyrosine kinases includes, Axl, Rse, and Mer. Axl plays a role in mediating cell growth and survival through apoptosis-mediated pathways and is thought to be up-regulated in melanomas. Breast tumor kinase (Brk) is a nonreceptor tyrosine kinase that is overexpressed in many breast and colon cancers. Like c-Src, overexpression of Brk leads to sensitization to EGF.

Calcium/calmodulin-dependent protein kinase-II (CaMKII) is a serine/threonine protein kinase involved in cardiac hypertrophy and heart failure. Casein kinases (CK) are ubiquitous serine/threonine kinases that are constitutively active. CKI and CKII have been implicated in Alzheimer's disease progression. Cyclin-dependent kinases (cdk) are proline-directed serine/threonine kinases that when mutated or over-expressed, can cause uncontrolled proliferation and tumorigenesis. Interest in their role in neurodegerative diseases such as Alzheimer's disease and Amyotrophic Lateral Sclerosis, in particular cdk5, is growing due to their role in the development of the central nervous system during embryogenesis.

The product of the c-kit proto-oncogene (c-Kit) is a tyrosine kinase receptor for stem cell factor. Ligand binding and activation of the receptor is critical for early stem cell differentiation in haematopoiesis and gametogenesis and melanogenesis. The D816H mutation has been shown to constitutively activate the protein and has been found in patients with gastrointestinal stromal tumors and mast cell leukemia. This mutation has also been shown to confer resistance to the kinase inhibitor Gleevec®. The V560G substitution is a somatic mutation associated with some gastrointestinal stromal tumors (GISTs). This mutation lies within the juxtamembrane region of the protein; mutations in this region of c-Kit have been found to be present in >50% of GISTs. Activating or gain-of-function mutations in the c-kit gene have been identified in many gastrointestinal stromal tumors (GISTs).

Death-associated protein kinase-1 (DAPK1) is a calcium/calmodulin-dependent serine/threonine kinase of the CAMK subfamily. Recent studies have shown that DAPK1 protein expression is reduced or silenced in some carcinoma cells by CpG methylation of the DAPK1 gene promoter region. Aberrant expression and signaling of discoidin domain tyrosine kinase receptors 1 and 2 (DDR1 and DDR2) have been implicated in tumor invasion, atherosclerosis and liver fibrosis through its ability to influence extracellular maxtrix remodeling. EGFR family members heterodimerize with each other to activate downstream signaling pathways and are aberrantly expressed in many cancers, such as breast cancer.

Fer is a non-receptor tyrosine kinase that has been implicated in inflammation and prostate cancer. Fes is a non-receptor tyrosine kinase with close homology to Fer. Fes is expressed in myeloid hematopoietic cells and plays a role in their differentiation. Aberrant expression of Fes is shown in breast and prostate cancer. Fibroblast growth factor receptor (FGFR) is a receptor tyrosine kinase. Mutations in this receptor can result in constitutive activation through receptor dimerization, kinase activation, and increased affinity for FGF. FGFR has been implicated in achondroplasia, angiogenesis, and congenital diseases.

Fms-like tyrosine kinase-4 (Flt4) is also known as VEGFR-3, and is predominantly expressed in adult lymphatic endothelium. It mediates both angiogenesis and lymphangiogenesis in tumors, and appears to play a role in tumor metastasis via the lymphatics. Insulin-like growth factors (IGF) I is a tyrosine kinase receptor that is activated by both IGF I and II. The IGF system is involved in skeletal growth, and is essential for the prevention of apoptosis in most cells. Strong evidence emphasizes the role of the IGF-IR signaling in tumorigenesis. The multi-subunit protein kinase, IKB kinase (IKK) is a serine/threonine kinase that is considered the master regulator of NFKB-mediated inflammatory responses. Inhibition of IKK activity can prevent the upregulation of various proinflammatory genes, thereby reducing inflammation. In addition to inflammatory diseases such as rheumatoid arthritis, IKK has also been implicated in cancer and diabetes.

The insulin receptor is a tyrosine kinase receptor that, when bound to insulin, initiates multiple signal transduction pathways, including activation of JNK, PI 3-kinase, Akt, and PKC. Pharmacological intervention of these insulin receptor-dependent pathways is of interest for the treatment of insulin resistance, obesity, and diabetes. The stress-activated protein kinase 1 (SAPK) family is also referred to as the jun N-terminal kinase family in light of the substrate preference of these serine/threonine kinases and has been implicated in many neurodegenerative diseases including Alzheimer's disease, Parkinson's disease, and Amyotrophic lateral sclerosis.

LIM kinase (LIMK) is a serine/threonine kinase known to play a role in the cognitive function. Misregulation of LIMK activity has resulted in cytoskeletal defects associated with Williams Syndrome, a neurodevelopmental disorder. There are three categories of MAPKs: c-Jun NH2-terminal kinases (JNKs), p38 MAPK, and extracellular signal-related kinases (Erks). Because of their role in mediating cellular processes, MAPK/Erks are key targets for anti-cancer therapies. Met is a tyrosine kinase receptor for Hepatocyte Growth Factor (HGF), thought to stimulate multiple cellular processes including cell proliferation, differentiation, cell migration and tumorigenesis. Chronic stimulation of Met on cancer cells is thought to play a role in metastasis.

The product of the mer proto-oncogene (Mer) is a transmembrane protein belonging to the Mer/Axl/Tyro3 receptor tyrosine kinase family. Although not detected in normal lymphocytes, Mer is expressed in B- and T-cell leukemia cell lines, suggesting an association with lymphoid malignancies. Phosphorylase kinase (PhK) is a heterotetrameric protein that mediates the neural and hormonal regulation of glycogen breakdown by glycogen phosphorylase. Heritable deficiency of PhK is responsible for 25% of all cases of glycogen storage disease and occurs with a frequency of 1 in 100,000 births.

Phosphatidylinositol (PI) 3-kinase is a serine/threonine protein kinase linked to numerous disease states, including allergic response, cancer, hypertension, atherosclerosis and inflammatory diseases. PIM kinases are serine/threonine protein kinases thought to be involved in regulating apoptosis, cell cycle progression and transcription by modulating various targets, including HSP90, STAT3 and STAT5. Elevated levels of Pim-1 expression have been observed in prostate cancer.

Protein kinase A (PKA) is a serine/threonine kinase activated by the second messenger cyclic AMP. Mutations in one of the subunits of the PKA holoenzyme is thought to cause Carney complex (CNC) and primary pigmented nodular adrenocortical disease (PPNAD). Protein kinase C enzymes belong to a family of serine/threonine kinases that fall into three general categories: conventional (PKC α, βI, βII, γ) isoforms that require calcium and diacylclycerol (DAG) for activity; novel (δ, ε, h, m, q) isoforms that are calcium-independent; and atypical (l, x) isoforms that are calcium and DAG-independent. PKC isozymes play an important role in cell proliferation and apoptosis in many cancers, including prostate cancer. PKD2 is the major isoform of the PKD family expressed in chronic myeloid leukemia cells and is tyrosine phosphorylated by Bcr-Abl in its pleckstrin homology domain.

The double-stranded RNA-activated protein kinase (PKR) is a serine/threonine kinase that modulates protein synthesis through the phosphorylation of translation initiation factor eIF-2a. PKR has been linked to numerous signal transduction pathways including caspase-8, JNK, p38 MAPK, and NF-κB. PKR hyperactivity has been linked to neurodegenerative diseases, such as Huntington disease, Alzheimer disease, and Amyotrophic Lateral Sclerosis.

The Raf proteins (Raf-1, A-Raf, B-Raf) are serine/threonine kinases that bind to activated Ras, resulting in their translocation to the plasma membrane, and subsequent activation. Inhibitors of Raf are of pharmacological importance, designed to block the Raf/MEK/ERK signaling pathway hyperactivated in many cancer tumor cell lines.

Ret is a tyrosine kinase receptor involved in the activation of several signaling pathways including the PLC gamma, Ras, JNK and inositol phosphate pathways. Ret mutations have been shown to be causative in several diseases, including Hirschsprung's disease (HD), papillary thyroid carcinoma, and multiple endocrine neoplasia (MEN) 2A, MEN 2B, and familial medullary thyroid carcinoma.

P70 S6 kinase is a serine/threonine kinase which phosphorylates the 40S ribosomal protein S6, and several translation-regulatory factors. It is thought to mediate cell-cycle progression and survival. Overexpression of S6 kinase has been observed in breast cancer and Alzheimer's disease. pp 60c-Src is a non-receptor tyrosine kinase over-expressed in several epithelial and non-epithelial cancers. Its role in cell division, motility, angiogenesis and survival has made c-Src an ideal target for cancer therapy.

TGF-β activated kinase (TAK1) is a member of the serine/threonine MAPKKK family and its kinase activity is stimulated in response to TGF-β, bone morphogenic protein (BMP) and ceramide. TAK1 can play a role in the pathophysiology of renal tubular disease and lung cancer. The Trk family of receptor tyrosine kinases include Trk A, Trk B, and Trk C. Trk receptors are thought to be excellent targets for cancer therapy.

Yes is a member of the Src family of non-receptor tyrosine kinases. Expression of Yes is elevated in melanocytes and in melanoma cells, and Yes kinase activity is stimulated by neurotrophins, which are mitogenic and metastatic factors for melanoma cells. In addition to melanoma, Yes is also over-expressed in colon cancer. Finally, ZAP-70 is a non-receptor tyrosine kinase of the Syk family, identified as a biomarker for Chronic Lymphocytic Leukemia (CLL) prognosis.

Today, with more than 500 protein kinases identified in the human genome, research has focused on understanding the molecular details of the roles kinases play in regulating critical cellular activities. More than 50 protein kinase inhibitors for cancer are in clinical testing or approved by the US Food and Drug Administration (FDA), including the blockbuster drugs Gleevec® (imatinib mesylate), Iressa® (gefitinib), and Tarceva® (erlotinib). These drugs have proven effective in blocking the action of their respective kinase target, without causing the negative side effects of traditional chemotherapy.

Further characterization of the central role that kinases play in disease and health, and development of new kinase-related diagnostic tests and therapeutics are ongoing areas of research. New tools for facilitating this research would contribute to myriad aspects of our understanding of kinases and their application in the medical sciences.

### 3. SUMMARY OF THE INVENTION

In one aspect, the invention provides a method for analysis of proteins in a sample comprising: a) contacting the sample with a first protein capture agent; b) separating the proteins bound to the first protein capture agent from the sample; c) digesting the proteins bound to the first protein capture agent with a protease to provide protein fragments having a scissile bond; and d) analyzing the products of the protease digestion by mass spectrometry.

In one embodiment, the first protein capture agent is a kinase capture agent, for example a non-selective kinase capture agent. The kinase capture agent can be a kinase inhibitor. In another embodiment, the first protein capture agent is a phosphatase capture agent.

In some embodiments, the protein capture agent, the kinase capture agent, and/or the phosphatase capture agent is labeled with a first member of an affinity pair, for example biotin.

In one embodiment, the method further comprises: a) contacting the sample with a second protein capture agent; b) separating the proteins bound to the second protein capture agent from the sample; and c) digesting the proteins bound to the second protein capture agent with a protease to provide protein fragments comprising a scissile bond.

In one embodiment the first protein capture gent is a kinase capture agent and the second protein capture agent is a kinase capture agent different from the first kinase capture agent.

In another embodiment, the first protein capture agent is a kinase capture agent and the second protein capture agent is a phosphatase capture agent.

In yet another embodiment, the method further comprises: a) providing to protein fragments having a scissile bond a calibrator peptide having a scissile bond and having the same amino acid composition and same mass as a protein fragment after protease digestion, wherein the calibrator peptide has a scissile bond in a different location from the protein fragment; and b) analyzing the calibrator peptide by mass spectroscopy.

In another aspect, the invention provides a method for analysis of proteins from a plurality of samples comprising: a) contacting each sample with a protein capture agent; b) separating the proteins bound to the protein capture agent from each sample; c) coupling a set of isobaric mass tags to the captured proteins or protein fragments, wherein proteins in each sample are coupled with a different isobaric mass tag from the set and wherein each isobaric mass tag in the set has a scissile bond in a different position than any other mass tag in the set; d) digesting the captured proteins with a protease to provide protein fragments; and e) detecting a plurality of isobaric mass tags by mass spectrometry in the same experiment.

In one embodiment, the isobaric mass tags are coupled to the captured proteins prior to digestion with a protease. In another embodiment, the isobaric mass tags are coupled to the protein fragments resultant from the digestion of captured proteins with a protease.

In one embodiment, each isobaric mass tag comprises a peptide. The scissile bond can be Asp-Pro bond.

In one embodiment, the method further comprises: a) providing to the protein fragments a calibrator peptide having a scissile bond and having the same amino acid composition and same mass as each isobaric mass tag in the set, wherein the calibrator peptide has a scissile bond in a different location from every isobaric mass tag in the set; b) detecting the calibrator peptide by mass spectrometry; and c) quantitatively correlating the mass spectrometry signals from the mass tag with the mass spectrometry signals from the calibrator peptide.

In another aspect, the invention provides a method for isolating a plurality of proteins from a sample comprising: a) providing a first kinase capture agent and a second protein capture agent; b) contacting the sample with the first kinase capture agent and the second protein capture agent; c) separating the proteins bound to the first kinase capture agent and the second protein capture agent from the sample.

In some embodiments, the first kinase capture agent is a non-selective kinase capture agent. In some embodiments, the second protein capture agent is a second kinase capture agent different from the first kinase capture agent.

In another aspect, the invention provides a kit comprising: a) a capture agent labeled with a first member of an affinity pair; b) a plate having one or more wells, wherein each well is coated with a second member of the affinity pair; and c) a set of instructions for use.

In some embodiments, the plate has 2, 4, 8, 16, 64, 96, 128, 256, 384 or 512 wells.

In some embodiments, the kit further comprises a set of calibrator peptides an/or set of isobaric mass tags.

In one embodiment, the first member of the affinity pair is biotin and the second member of the affinity pair is streptavidin.

In one embodiment the capture agent is a kinase capture agent or a phosphatase capture agent.

Exemplary kinase capture agents include, but are not limited to, kinase inhibitors. Exemplary kinase inhibitors include staurosporine or a staurosporine analogs. Staurosporine analogs include, but are not limited to 7-hydroxystaurosporine, N-benzoylstaurosporine, 3-hydroxy-4′-N-methylstaurosporine, 3-hydroxy-4′-N-demethylstaurosporine, 3′-demethoxy-3′-hydroxy-4′-N-demethylstaurosporine, staurosporine aglycone or 4′-N-benzoyl staurosporine. Further exemplary kinase inhibitors include, but are not limited to, KT 5720, K252, H-9, rottlerin, quercetin, hymenialdisine, SB 203580, myricetin, SU11248, roscovitine, EKB569, or SB202190.

Exemplary phosphatase capture agents include, but are not limited to, phosphatase inhibitors. Exemplary phosphatase inhibitors include, but are not limited to, okadaic acid, tautomycin, microcystin, a microcystin derivative, calyculin A, calyculin B, calyculin C, calyculin D, calyculin E, calyculin F, calyculin G, calyculin H, cantharidin, thyrsferyl 23-acetate, isopalinurin, dragacidin, a dragacidin derivative, fostriecin, 1-(oxalyl-amino)-4,5,6,7-tetrahydro-thieno[2,3-c]pyridine-3-carboxylic acid or bis-(maltoato)-oxovanadium(IV).

In one embodiment, the protease is trypsin.

In one embodiment, mass spectroscopy is tandem mass spectroscopy.

### 4. BRIEF DESCRIPTION OF DRAWINGS

FIGS. 1A-D show staurosporine and some of its commercially available analogs;

FIG. 2 shows a staurosporine analog suitable for immobilization on solid-phase supports;

FIG. 3 shows an example of tandem mass spectrometry-based quantification of HER2 kinase; and

FIG. 4 shows exemplary isobaric mass tags and the analytical signals derived from them in tandem mass spectrometry.

### 5. DETAILED DESCRIPTION OF THE INVENTION

### 5.1 Definitions

As used herein, an “affinity pair” refers to a pair of molecules that exhibit strong non-covalent interaction. Affinity pairs include, but are not limited to, biotin-avidin, biotin-streptavidin, heavy metal derivative-thio group, various homopolynucleotides such as poly dG-poly dC, polydA-poly dT and poly dA-poly dU, various oligonucleotides of specific sequences (where the analyte of interest comprises a nucleic acid sequence that hybridizes to the oligonucleotide), and antigen (or epitopes thereof)-antibody pairs.

As used herein, by “couple” or “coupling” is meant forming a covalent or non-covalent (e.g., ionic or hydrogen) chemical bond.

As used herein, a “scissile bond” is also meant to encompass a “sessile bond.”

As used herein, “isobaric tag” means a tag having the same total mass as a protein fragment and/or a tag having the same total mass as another tag. In some embodiments, isobaric tags become non-isobaric during analysis by mass spectrometry.

As used herein, “non-selective capture agent” means a capture agent that can capture a variety of different proteins of the same protein group. For example, a “non-selective kinase capture agent” can capture a variety of different kinases. In some embodiments, a non-selective kinase capture agent captures 50% or more, 60% or more, 70% or more, 80% or more, 90% or more, or 95% or more of kinases.

As used herein, “quantify” and “quantitate” are meant to be synonyms.

As used herein, the term “analyzing” includes qualitatively detecting, quantitatively detecting or qualitatively and quantitatively detecting.

### 5.2 Kinases and Phosphatases

The kinome is a subset of the genome consisting of the protein kinase genes. The complete complement of over 500 protein kinases constitutes one of the largest of all human gene families. Protein kinases act as key regulators of cell function by catalyzing the addition of a negatively charged phosphomonoester group to proteins. This process of protein phosphorylation, in turn, regulates protein function in both normal and disease states.

The technology described herein provides methods for enriching (or isolating) kinases, for example ATP-dependent kinases, utilizing one or more kinase capture agents. Examples of kinase capture agents include, but are not limited to, relatively non-selective protein kinase inhibitors, substrates or pseudosubstrates. The methods are useful, for example, for profiling of kinomes by tandem mass spectrometry. Although many highly selective and potent small molecule kinase inhibitors have been previously identified, as is described herein above, a large number of relatively non-selective small molecule kinase inhibitors have also been identified. For the methods described herein, use of relatively non-selective small molecule kinase inhibitors reduces the need for tailoring purification procedures for individual kinases, and amplifies the analytical signal obtained by enriching enzymes normally present in cells, tissues and bodily fluids at only catalytic concentrations. However, it will be recognized that selective small molecule kinase inhibitors also can be useful in these kinase analysis methods. In addition, a combination of a non-selective and a selective small molecule kinase inhibitor can be useful in these methods. Furthermore, a kinase capture agent (or more than one kinase caputure agent) can also be combined with a phosphatase capture agent to enrich (or isolate) kinases and phosphatases concurrently.

The methods described herein also can be applied to multiplexed analysis of protein kinases and/or phosphatases by tandem mass spectrometry from a single or multiple specimens.

In one embodiment, the technology described herein provides a method for analyzing a population of kinases, such as a kinome. The method involves separating kinases from a sample using one or more kinase capture agents, proteolytically digesting a protein sample to constituent peptides (for example with a protease such as trypsin), supplementing the obtained peptides with rationally designed calibrator peptides relating to particular protein kinase peptide sequences that contain scissile aspartate-proline (DP) bonds, and quantifying the native peptides derived from the kinase population by tandem mass spectrometry. Strategies for profiling the relative abundance of protein and lipid kinases in multiple samples using isobaric peptide tags containing scissile DP bonds are also described. One of skill in the art will recognize that similar methodology can be applied to analyze phosphatases or a combination of kinases and phosphatases.

### 5.3 Use of Kinase Inhibitors as Kinase Capture Agents

ATP is a cofactor for the protein and lipid kinase families of enzymes. Previous studies have shown that, when bound to adenosine cyclic 3′,5′-monophosphate-dependent protein kinase (cAMP kinase), the adenosine portion of ATP is buried deep within the catalytic cleft of the kinase, with the alpha, beta and gamma phosphate residues protruding towards the opening of the cleft. The unique spatial positioning of ATP within the catalytic cleft of this model kinase and its interactions with conserved amino acids found in all protein kinases renders ATP a useful affinity ligand for the enrichment of the entire protein kinase family. Thus, adenosine-5′-(gamma-4-aminophenyl) triphosphate has been covalently linked to solid phase supports, such as Sepharose, through its gamma phosphate. As an example, adenosine-5′-(gamma-4-aminophenyl) triphosphate-Sepharose has been used as an affinity matrix. Non-hydrolyzable analogs of ATP, such as adenosine 5′-O-(3-thio) triphosphate (ATPgammaS) also have been used for this purpose, and provide increased stability as affinity matrices. Numerous other ATP-binding proteins, such as pyruvate kinase and hexokinase, have been similarly enriched by this basic approach.

The majority of small molecule protein kinase inhibitors target the ATP-binding pocket of the enzymes. These inhibitors generally are characterized as quinazolines, pyrimidines, flavonoids, paullones or alkaloids. The non-selective small molecule organic ATP-mimetic inhibitors, targeting the ATP-binding pocket, tend to interact with a variety of different protein kinases. For example, the fungal alkaloid staurosporine, despite a seemingly unrelated chemical structure, binds with the same key hydrogen-bond interactions as ATP in its binding mode. The heterocyclic ring system of staurosporine is almost congruent to the adenyl group of ATP, the lactam generates the same hydrogen bonds as the adenine residue to the enzyme, and the N-glycosyl-group is bound in the ribose pocket. This has led some to comment on the inhibitor's poor selectivity profile: “The superimposition of ATP and staurosporine shows that the inhibitor is simply “too good” at imitating ATP.” (Huwe A, Mazitschek R. and Giannis, A. Small molecules as inhibitors of cyclin-dependent kinases. Angew. Chem. Int. Ed. Engl. 2003 May 16; 42(19):2122-38).

In an embodiment, the methods described herein employ the kinase inhibitor staurosporine as a non-selective kinase capture agent. Staurosporine binds to a broad-spectrum of protein kinases with an affinity that is actually higher than ATP itself. Staurosporine inhibits most protein kinases at low nanomolar concentrations, in a competitive manner with respect to ATP and can be considered a prototypical broad-spectrum small molecule inhibitor for enrichment of kinase populations prior to MS/MS analysis. Fortunately, the majority of the small ATP-mimetic inhibitors, routinely used as protein kinase inhibitors, do not seem to interact broadly with other ATP-binding enzymes, such as the intermediary metabolism enzymes, hexokinase and pyruvate kinase. Other indolocarbazole alkaloids, such as 7-hydroxystaurosporine (UCN-01), (Sigma-Aldrich, St. Louis, Mo.), N-benzoylstaurosporine (CGP 41251), 3-hydroxy-4′-N-methylstaurosporine, 3-hydroxy-4′-N-demethylstaurosporine, and 3′-demethoxy-3′-hydroxy-4′-N-demethylstaurosporine can also be useful for enrichment of kinase populations, such as significant portions of the kinome. Examples of other broad-spectrum small molecule organic protein kinase inhibitors suitable for the methods described herein include KT 5720 (Sigma-Aldrich, St. Louis, Mo.), K252a and K525b (Fermentek, Jerusalem, Israel), H-9 (EMD Chemicals Inc., an Affiliate of Merck KGaA, Darmstadt, Germany), rottlerin (Millipore, Billerica, Mass.), quercetin (Sigma-Aldrich, St. Louis, Mo.), hymenialdisine (BIOMOL International, Plymouth Meeting, Pa.), SB 203580 (A.G. Scientific, Inc, San Diego, Calif.) and myricetin (Sigma-Aldrich, St. Louis, Mo.). In some embodiments, small molecule kinase inhibitors possess enzyme binding constants that are much lower than ATP, bind to kinases in a magnesium-independent fashion, not interact significantly with nucleotide-requiring intermediary metabolism enzymes, and associate directly with the ATP-binding site of protein and lipid kinases. The choice of small molecule inhibitors need not be constrained to cell-permeant molecules for the methods described herein.

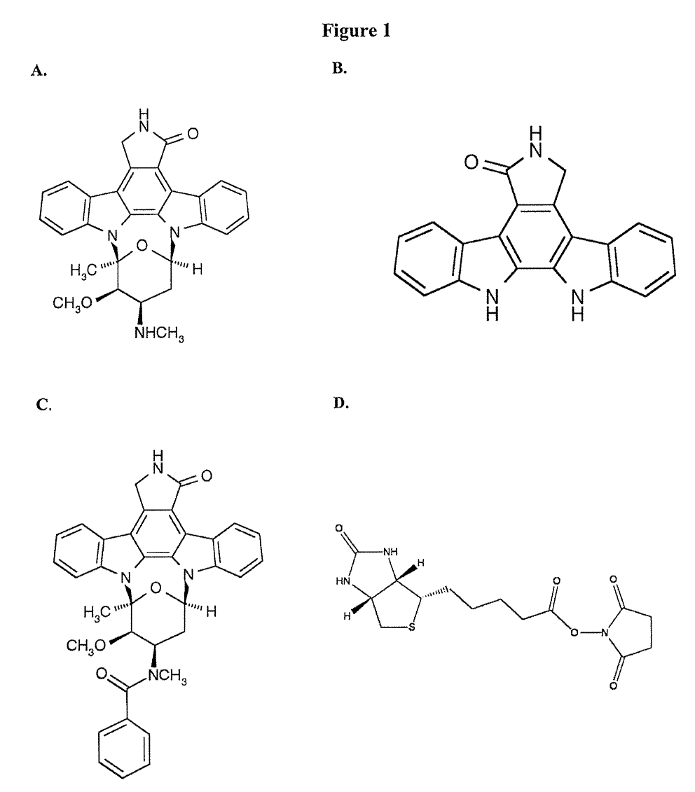

FIG. 1 shows the structure of staurosporine (A) and two chemically-related protein kinase inhibitors, staurosporine aglycone (also known as K252c) (B), and 4′-N-benzoyl staurosporine (CGP 41251) (C), all of which are commercially available from LC Laboratories, Woburn, Mass. The ability of staurosporine aglycone to inhibit protein kinases suggests that the glycone portion of staurosporine can be targeted for attachment of linkers or tags, without substantially perturbing interaction with the ATP binding pocket of protein kinases. The activity of 4′-N-benzoyl staurosporine further demonstrates that linkers or tags can be affixed at the secondary amine position of the methylamine (R—NH—CH₃) portion of the inhibitor without interfering substantially with kinase binding. Furthermore, numerous high resolution X-ray crystallography studies of different protein kinases complexed with staurosporine support the concept that the glycone ring partially protrudes from the opening of the nucleotide-binding cleft and is suitable for affixing tags or linkers. Thus, biotinylation (or attachment of another molecule that is a member of an affinity pair) is targeted to this portion of staurosporine and the product then immobilized on streptavidin-coated multi-well plates (i.e., coated with a second member of an affinity pair), magnetic beads, stacked filters, MALDI target plates or other solid phase substrates, in order to serve as an affinity capture substrate for many members of the kinome. N-hydroxysuccinimidyl (NHS) esters of biotin, shown, for example in FIG. 1D (commercially available from Quanta BioDesign, Powell, Ohio), can react directly with the secondary amine of staurosporine (for exemplary protocol see Bioconjugate Techniques by Greg T. Hermanson, Academic Press, 1996, San Diego, Calif., incorporated by reference herein), creating a stable imide linkage and thus facilitate attachment of the molecule to streptavidin-coated substrates. Other suitable members of affinity pairs can also be useful for this purpose.

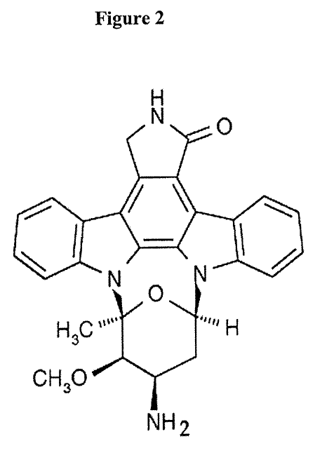

Alternatively, the compound shown in FIG. 2 is readily biotinylated using a reagent such as biotin-PEG-NHS reagent, commercially available from Nektar Pharmaceuticals (San Carlos, Calif.), creating a stable amide linkage. Also, the compound in FIG. 2 can be PEGylated using similar chemistries and directly immobilized on epoxy-activated surfaces by standard chemistry. Numerous other methods for linking the small molecule kinase inhibitors to solid-phase substrates are available and well known to those skilled in the art. For example, many procedures are described in Bioconjugate Techniques by Greg T. Hermanson, Academic Press, 1996, San Diego, Calif.

Once a small molecule organic kinase inhibitor labeled with a first member of an affinity pair, such as biotinylated staurosporine, has been immobilized on a suitable solid phase support coated with a second member of an affinity pair, such as a streptavidin-coated 96-well plate, the solid phase substrate can be blocked with excess biotin and washed with a blocking buffer, such as 1% bovine serum albumin, 0.05% Tween-20 detergent and 1 mM dithiothreitol (DTT) in phosphate-buffered saline. Next, cellular lysates can be prepared in 0.05% Tween-20 detergent and 1 mM DTT in phosphate-buffered saline, centrifuged at 6,000×g and filtered (0.2 μm), in order to remove cellular debris. Then, the clarified cellular lysates are incubated in the wells of the plate, and subsequently washed extensively to remove proteins that do not associate with staurosporine. Stringency of binding can be controlled by systematically varying ionic strength in the incubation and wash buffer using, for example, 20 mM to 4 M NaCl or 150 mM to 1 M NaCl. The resulting enriched protein kinase sample is then subjected to proteolytic digestion using an enzyme, such as trypsin, and the resulting peptides recovered for further analysis. Unlike conventional affinity chromatography methods, defining elution conditions for the protein kinases is not necessary, since they are proteolytically removed as an integral step of the analysis procedure. Furthermore, retention of catalytic activity is immaterial to the profiling method.

In some embodiments, the analysis involves filtering of isobaric mass tags (and the attached protein fragments), protein fragments and/or calibrator from other molecules based on mass-to-charge ratio, fragmentation of the scissile (DP) bond to provide fragments having different masses, and detection of the different fragments based on their mass-to-charge ratios. The first stage filtering can be used to produce predetermined patterns that indicate whether the second, fragmentation stage should be performed and/or which portion(s) of the analyzed material can or should be analyzed in the fragmentation stage.

In some embodiments, the analysis carried out using a tandem mass. The same sample can be analyzed both with and without fragmentation (by operating with and without collision gas), and the results compared to detect shifts in mass-to-charge ratio. Both the unfragmented and fragmented results should give diagnostic peaks, with the combination of peaks both with and without fragmentation confirming the mass tag (and corresponding sample), protein fragment, or calibrator peptide involved. In one embodiment, such distinctions are accomplished by using appropriate sets of isobaric mass tags and allow large scale multiplexing in the detection of analytes.

The analysis and/or detection steps of the disclosed methods can be performed with a MALDI-QqTOF mass spectrometer. The method enables a multiplexed analyte detection, and high sensitivity. Useful tandem mass spectrometers are described by Loboda et al., Design and Performance of a MALDI-QqTOF Mass Spectrometer, in 47th ASMS Conference, Dallas, Tex. (1999), Loboda et al., Rapid Comm. Mass Spectrom. 14(12):1047-1057 (2000), Shevchenko et al., Anal. Chem., 72: 2132-2142 (2000), and Krutchinsky et al., J. Am. Soc. Mass Spectrom., 11(6):493-504 (2000). In such an instrument the sample is ionized in the source (MALDI, for example) to produce charged ions; it is useful if the ionization conditions are such that primarily a singly charged parent ion is produced. First and third quadrupoles, Q0 and Q2, will be operated in RF only mode and will act as ion guides for all charged particles, second quadrupole Q1 will be operated in RF+DC mode to pass only a particular mass-to-charge (or, in practice, a narrow mass-to-charge range). This quadrupole selects the mass-to-charge ratio, (m/z), of interest. The collision cell surrounding Q2 can be filled to appropriate pressure with a gas to fracture the input ions by collisionally induced dissociation (normally the collision gas is chemically inert, but reactive gases are contemplated). In some embodiments, a scissile bond is preferentially fractured in the Q2 collision cell.

A MALDI source is useful for the disclosed method because it facilitates the multiplexed analysis of samples from heterogeneous environments such as arrays, beads, microfabricated devices, tissue samples, and the like. An example of such an instrument is described by Qin et al., A practical ion trap mass spectrometer for the analysis of peptides by matrix-assisted laser desorption/ionization., Anal. Chem., 68:1784-1791 (1996.

A number of elements contribute to the sensitivity of the disclosed method. The filter quadrupole, Q1, selects a narrow mass-to-charge ratio and discriminates against other mass-to-charge ions, significantly decreasing background from non germane ions. For example, for a sample containing a distribution of mass-to-charges of width 3000 Da, a mass-to-charge transmission window of 2 Da applied to this distribution can improve the signal to noise by at least a factor of 3000/2=1500. Once the parent ion is selected by quadrupole Q1, fragmentation of the parent ion, for example into a single charged daughter ion, has the advantage over systems which fragment the parent into a number of daughter ions. For example, a parent fragmented into 20 daughter ions will yield signals that are on average 1/20th the intensity of the parent ions. For a parent to single daughter system there will not be this signal dilution.

A useful system for use with the disclosed method has a high duty cycle, and as such good statistics can be collected quickly. For the case where a single set of isobaric mass tags is used, the multiplexed detection is accomplished without having to scan the filter quadrupole (although such a scan is useful for single pass analysis of a complex protein sample with multiple labeled proteins). MALDI sources can operate at several kHz, quadrupoles operate continuously, and time of flight analyzers can capture the entire mass-to-charge region of interest at several kHz repetition rate. Thus, the overall system can acquire thousands of measurements per second. For throughput advantage in a multiplexed assay the time of flight analyzer has an advantage over a quadruple analyzer for the final stage because the time of flight analyzer detects all fragment ions in the same acquisition rather than requiring scanning (or stepping) over the ions with a quadrupole analyzer.

The disclosed methods are compatible with techniques involving cleavage, treatment, or fragmentation of a bulk sample in order to simplify the sample prior to introduction into the first stage of a multistage detection system. The disclosed method is also compatible with any desired sample, including raw extracts and fractionated samples.

While staurosporine is a potent inhibitor for at least 90% of known protein kinases, it is ineffective for a small percentage of them. For example, ERBB2, p38α, p38β, NEK6, PKMYT1, EPHB4, JAK1 and CSNK161 are examples of protein kinases that are not potently inhibited by staurosporine. In instances where the promiscuity of a kinase inhibitor is not sufficiently broad to cover particular kinases that are required in a particular kinome-wide analysis, it is feasible to supplement the primary capture agent with additional immobilized kinase inhibitors. By co-immobilizing two or more kinase inhibitors on the solid phase substrate, the combined capabilities of the individual inhibitors can be used to increase the comprehensiveness of kinome coverage. In the instance wherein staurosporine is used as the primary capture agent, SU11248 (sunitinib, marketed by Pfizer as SUTENT®) could be included as a secondary capture agent in order to recover JAK1 in the kinome profiling experiments. Roscovitine (CYC202), available from Sigma-Aldrich, could be employed in order to include CSNK1G1 in the profile, and EKB569 could be used in order to include EPHB4 and PKMYT1 in the profiling. SB203580 or SB202190 can be included to supplement kinome profiles with p38 protein kinases. Additionally, it is possible to restrict kinome coverage by using a more selective kinase inhibitor as the capture agent or by including soluble kinase inhibitors in the binding buffer to competitively inhibit binding of particular kinases to the more promiscuous kinase inhibitor bound to the solid phase substrate. For example, using staurosporine as a binding moiety for capturing kinases, and supplementing the reaction medium with soluble SU11248, would block binding of KIT, PDGFRB and VEGFR2 protein kinases.

### 5.4 Phosphatases

The regulation of protein phosphorylation requires coordinated control of both protein kinases and protein phosphatases. There are over 120 different protein phosphatases in the human genome. Three distinct classes of protein phosphatases are known; tyrosine-specific, serine/threonine-specific and dual-specificity phosphatases. The phosphatase classes can be further subdivided into various subtypes. For example, the serine/threonine-specific phosphatases are classified into four major subtypes, PP1, PP2, PP2B (calcineurin) and PP2C (ATP/Mg²⁺-dependent protein phosphatase). Multiple isoforms of each of the subtypes also exist such as PP4 (related to PP1), PP5 (similar to PP1, PP2A, PP2B, PP4) PP6 (similar to PP5), PP7 (similar to all major classes of phosphatase), PPZ1 (PP1 relative), PPZ2 (PP1 relative), PPQ (PP1 relative), PPV(PP2A relative), PPG (PP2A relative) and rdgc (PP2B relative). The regulation of phosphatases is thought to be as complex as that of kinases and it makes sense to assay both classes of enzymes when comprehensively evaluating signaling pathways. Natural product-derived inhibitors of protein phosphatases are known, such as the potent competitive inhibitors of both PP1 and PP2A, such as okadaic acid, tautomycin, the microcystins, and calyculins A-H. Additionally, a variety of other, more selective inhibitors of protein phosphatases have been uncovered including cantharidin, thyrsferyl 23-acetate, isopalinurin, dragacidins and fostriecin.

When profiling the kinome, it is feasible to simultaneously profile protein phosphatases by co-immobilizing a protein phosphatase capture agent (e.g., a phosphatase inhibitor) with a protein kinase capture agent (e.g., a kinase inhibitor). For use in the methods described herein, a protein phosphatase inhibitor generally inhibits a broad range of protein phosphatases, without interacting significantly with other metabolic enzymes, such as mitochondrial pyruvate dehydrogenase phosphatase, acid phosphatases and alkaline phosphatases. Protein phosphatases can be proteolytically digested as an integral step of the analysis procedure. It is not necessary that enzymes retain catalytic activity during this process.

One exemplary protein phosphatase inhibitor suitable for the methods described herein is the monocyclic heptapeptide, microcystin. Methods for biotinylating microcystin are well known. Typically, the N-methyldehydroalanine residue of microcystin is derivatized with ethanedithiol. The reaction product is then combined with iodoacetyl-LC-biotin (Pierce Chemical, Rockford, Ill.). The final product can be further purified by preparative reverse-phase high-performance liquid chromatography, evaporation to dryness and stored in neat ethanol at −20° C. before use. The microcystin-biotin and staurosporine-biotin can then be simultaneously immobilized on a streptavidin-coated substrate, creating a matrix that simultaneously enriches kinases and serine/threonine phosphatases. Using similar strategies, protein tyrosine phosphatases can be included in the kinome-wide screen. For example, the selective PTP1B inhibitor 2-(oxalyl-amino)-4,5,6,7-tetrahydro-thieno[2,3-c]pyridine-3-carboxylic acid (OTP) can be coupled to epoxy-activated Sepharose 6B by standard methods, and then the beads mixed with streptavidin-agarose beads that have been pre-loaded with staurosporine-biotin to create a mixed matrix with wider target enzyme selectivity. Immobilized forms of nonspecific phosphotyrosine phosphatase inhibitors, such as bis-(maltolato)-oxovanadium(IV) can also be employed for expanding kinome coverage to protein phosphatase counterparts.

### 5.5 Quantitative Analyses of Protein Kinases and Phosphatases

In some embodiments, accurate quantitation of protein kinases and phosphatases is achieved by adding an internal standard of known concentration to the sample prior to analysis by mass spectrometry. Useful internal standards include calibrator peptides.

5.5.1. Calibrator Peptides

The technology described herein provides a multiplexed quantification strategy for the precise determination of protein kinase levels. In one embodiment, the method relies upon the use of synthetic internal standard peptides (calibrator peptides) that are introduced at known concentrations to enriched kinase samples prior to, during or after their proteolytic digestion. The synthetic calibrator peptides mimic native DP-containing peptide sequences within specific kinases, produced during proteolysis of the target proteins, except that amino acid sequences are rationally rearranged relative to the aspartate-proline (DP) bond. Thus, a calibrator peptide will have the same amino acid composition and same mass as a kinase fragment produced during proteolytic digestion of the kinase, but a different amino acid sequence. In some embodiments, one or more calibrator peptides may be used.

No stable isotopically labeled amino acids are required in the calibrator peptides, which makes them economical to manufacture. Analysis of the proteolyzed sample in a tandem mass spectrometer results in the direct detection and quantification of both the native peptides and the rationally-scrambled calibrator peptides. The simplicity and sensitivity of the method, coupled with the widespread availability of tandem mass spectrometers, make the strategy a useful procedure for measuring the levels of multiple kinases directly from the enriched kinase population.

The absolute quantification method is based upon the observation that a significant percentage of protein and lipid kinases contain at least one scissile DP bond. It should be noted, however, that other protein and lipid kinases do not contain this labile bond and they are not suitable targets for the absolute quantification method described herein. However, these kinases can be evaluated using the relative quantification method described herein below. Table I presents some representative protein and lipid kinases amenable to the absolute quantification approach outlined herein.

TABLE 1
Examples of protein and lipid kinases containing tryptic peptides with scissile
aspartate-proline (DP) bonds, highlighted in boldface:

| Protein ID | Peptide Sequence |
|---|---|
| gi\|178326\|gb\|AAA58364.1\|AKT2, protein | (R) APGEDPMDYK |
| serine/threonine kinase | |
| gi\|45331215\|ref\|NP_115805.1\| leucine zipper, | (R) EPPVPPATADPFLLAESDEAK |
| putative tumor suppressor | |
| gi\|25952118\|ref\|NP_741960.1\| calcium/calmodulin- | (K) MCDPGMTAFEPEALGNLVEGLDFHR |
| dependent protein kinase IIA isoform 2 [Homo | |
| sapiens] | |
| gi\|25952118\|ref\|NP_741960.1\| calcium/calmodulin- | (K) ICDPGLTSFEPEALGNLVEGMDFHR |
| dependent protein kinase IIB isoform 5 [Homo | |
| sapiens] | |
| gi\|26667183\|ref\|NP_742113.1\| calcium/calmodulin- | (K) ICDPGLTAFEPEALGNLVEGMDFHR |
| dependent protein kinase II delta isoform 1 [Homo | |
| sapiens] | |
| gi\|27437027\|ref\|NP_757380.1\| calcium/calmodulin- | (K) LVEVLDDPNEDHLYMVFELVNQGPVMEVPTLK |
| dependent protein kinase kinase 2 beta isoform 2 | |
| [Homo sapiens] | |
| gi\|4502613\|ref\|NP_001228.1\| cyclin A [Homo | (K) VESLAMFLGELSLIDADPYLK |
| sapiens] | |
| gi\|4826675\|ref\|NP_004926.11\| cyclin-dependent | (K) YFDSCNGDLDPEIVK |
| kinase 5 [Homo sapiens] | |
| gi\|4502623\|ref\|NP_001230.1\| cyclin H [Homo | (K) VLPNDPVFLEPHEEMTLCK |
| sapiens] | |
| gi\|38176158\|ref\|NP_003849.2\| cyclin K [Homo | (K) DLAHTPSQLEGLDPATEAR |
| sapiens] | |
| gi\|4502747\|ref\|NP_001252.1\| cyclin-dependent | (K) LLVLDPAQR |
| kinase 9 [Homo sapiens] | |
| | (R) IDSDDALNHDFFWSDPMPSDLK |
| gi\|6005850\|ref\|NP_009125.1\| protein kinase CHK2 | (R) EADPALNVETEIEILK |
| isoform a [Homo sapiens] | |
| gi\|67551261\|ref\|NP_004062.2\| CDC-like kinase 1 | (R) SEIQVLEHLNTTDPNSTFR |
| isoform 1 [Homo sapiens] | |
| | (K) MLEYDPAK |
| gi\|11177008\|dbj\|BAB17838.1\| casein kinase 1 | (K) EYIDPETK |
| gamma 1 [Homo sapiens] | |
| gi\|51873043\|ref\|NP_892027.2\| G protein-coupled | (R) LEANMLEPPFCPDPHAVYCK |
| receptor kinase 4 isoform alpha [Homo sapiens] | |
| gi\|89363047\|ref\|NP_004929.2\| death-associated | (R) LLDPPDPLGK |
| protein kinase 1 [Homo sapiens] | |
| gi\|49574532\|ref\|NP_063937.2\| glycogen synthase | (K) PQNLLVDPDTAVLK |
| kinase 3 alpha [Homo sapiens] | |
| gi\|20986531\|ref\|NP_620407. 1\| mitogen-activated | (R) VADPDHDHTGFLTEYVATR |
| protein kinase 1 [Homo sapiens] | |
| | (R) IEVEQALAHPYLEQYYDPSDEPIAEAPFK |
| gi\|23272546\|gb\|AAH35596.1\| p21(CDKN1A)- | (K) PVVDPSRITR |
| activated kinase 6 [Homo sapiens] | |
| | (R) AQSLGLLGDEHWATDPDMYLQSPQSER |
| | (R) TDPHGLYLSCNGGTPAGHK |
| | (R) TWHAQISTSNLYLPQDPTVAK |
| gi\|4506067\|ref\|NP_002728.1\| protein kinase C, | (K) NLIPMDPNGLSDPYVK |
| alpha [Homo sapiens] | |
| gi\|20149547\|ref\|NP_002944.2\| ribosomal protein S6 | (K) EPWPLMELVPLDPENGQTSGEEAGLQPSK |
| kinase, 90kDa, polypeptide 1 isoform a [Homo | |
| sapiens] | |
| | (K) ADPSHFELLK |
| | (K) MLHVDPHQR |
| gi\|25168263\|ref\|NP_005618.2\| serum/glucocorticoid | (R) HFDPEFTEEPVPNSIGK |
| regulated kinase [Homo sapiens] | |
| gi\|47419936\|ref\|NP_003128.3\| SFRS protein kinase | (R) NSDPNDPNR |
| 1 [Homo sapiens] | |
| gi\|5454094\|ref\|NP_006272.1\| serine/threonine | (K) ALDPMMER |
| kinase 3 (STE20 homolog, yeast) [Homo sapiens] | |
| gi\|4507917\|ref\|NP_003381.1\| weel tyrosine kinase | (K) VMIHPDPER |
| [Homo sapiens] | |
| gi\|2981233\|gb\|AAC06259.1\| mitotic checkpoint | (K) GNDPLGEWER |
| kinase Bub1 [Homo sapiens] | |
| gi\|9973390\|sp\|P57043\|ILK2_HUMAN Integrin- | (K) ICMNEDPAK |
| linked protein kinase 2 (ILK-2) | |
| gi\|3954946\|emb\|CAA74194.1\| PI-3 kinase [Homo | (K) QNADPSLISWDESGVDFYSK |
| sapiens] | |
| | (R) GLSGSDPTLNYNSLSPLEGPPNHSTSQGPQPGSDPWPK |
| | (K) LSFQNVDPLGENIRVIFK |
| | (R) GLQLLQDGNDPDPYVK |
| | (K) IYLLPDPQK |
| gi\|16506130\|dbj\|BAB70696.1\| phosphatidy- | (R) TDSASADPGNLK |
| linositol3-kinase-related protein kinase [Homo sapiens] | |
| | (K) LEGRDVDPNR |
| gi\|2827756\|sp\|P217091\|EPA1_HUMAN Ephrin type- | |
| A receptor 1 precursor (Tyrosine-protein kinase receptor EPH) | (K) PYVDLQAYEDPAQGALDFTR |
| | (R) ELDPAWLMVDTVIGEGEFGEVYR |
| gi\|3878441\|sp\|Q9H4B4\|CNK_HUMAN Cytokine | (R) GPELEMLAGLPTSDPGR |
| inducible serine/threonine-protein kinase (FGF- | |
| inducible kinase) (Proliferation-related kinase) | |
| gi\|21614496\|ref\|NP_006104.3\| vav3 oncogene | (K) HTTDPTEK |
| [Homo sapiens] | |
| | (K) QVDPGLPK |
| gi\|10862701\|ref\|NP_065681.1\| ret proto-oncogene | (K) CFCEPEDIQDPLCDELCR |
| isoform c; hydroxyaryl-protein kinase; cadherin | |
| family member 12; oncogene RET [Homo sapiens] | |
| gi\|7960243\|gb\|AAF71263.1\|AF2462191 SNARE | (K) AVNGAENDPFVR |
| protein kinase SNAK [Homo sapiens] | |
| | (R) FDVHQLANDPYLLPHMR |
| gi\|20380195\|gb\|AAH27984.1\| glycogen synthase | (K) PQNLLVDPDTAVLK |
| kinase 3 alpha [Homo sapiens] | |
| gi\|1154575\|ref\|NP_071331.1\|casein kinase 1, | (K) EYIDPETK |
| gamma 1 [Homo sapiens] | |
| gi\|4099129\|gb\|AAD09237.1\| AMP-activated protein | (K) FFVDGQWTHDPSEPIVTSQLGTVNNIIQVK |
| kinase beta subunit [Homo sapiens] | |
| | (K) DTGISCDPALLPEPNHVMLNHLYALSIK |
| gi\|2013725\|sp\|Q9UM73\| ALK_HUMAN ALK | (K) HYLNCSHCEVDECHMDPESHK |
| tyrosine kinase receptor precursor (Anaplastic | |
| lymphoma | |
| | (R) IEYCTQDPDVINTALPIEYGPLVEEEEK |
| gi\|21431788\|sp\|P27987\|IP3L_HUMAN 1D-myo- | (R) TLDPNSAFLHTLDQQK |
| inositol-trisphosphate 3-kinase B (Inositol 1,4,5- | |
| trisphosphate 3-kinase) (IP3 3-kinase) (IP3K-B) | |
| | (K) MIEVDPEAPTEEEK |
| gi\|306840\|gb\|AAA75493.1\| HER2 receptor | (R) GTQLFEDNYALAVLDNGDPLNNTTPVTGASPGGLR |
| | (K) GLQSLPTHDPSPLQR |

Table 2 illustrates examples of internal calibrants designed for the quantification of three different kinases, phosphoinositide 3-kinase (PI-3 kinase), an enzyme that phosphorylates the 3 position hydroxyl group of the inositol ring of phosphatidylinositol, ephrin type-A receptor (EPH), a protein-tyrosine kinase and HER2/neu (also known as ERBB-2), a member of the epidermal growth factor receptor (EGFR) family.

TABLE 2
Proteolytic fragments of exemplary protein and
lipid kinases and appropriate peptide calibrants:

| Protein | Native or | Amino Acid | Mass | | Mass |
|---|---|---|---|---|---|
| Kinase | Calibrant | Sequence | (Da) | Signal | (Da) |
| PI-3 Kinase | Native | (K)LSFQNVDPLGENIR | 1600.82 | PLGENIR | 697.44 |
| | Calibrant 1 | (K)LSNQNVDPLGEFIR | 1600.82 | PLGEFIR | 830.47 |
| | Calibrant 2 | (K)LSFNNVDPLGEQIR | 1600.82 | PLGEQIR | 811.46 |
| EPH | Native | (K)PYVDLQAYEDPAQGALDFTR | 2268.07 | PAQGALDFTR | 1074.55 |
| | Calibrant 1 | (K)PYFDLQAYEDPAQGALDVTR | 2268.07 | PAQGALDVTR | 1026.55 |
| | Calibrant 2 | (K)PYVDLQGYEDPAQAALDFTR | 2268.07 | PAQAALDFTR | 1088.56 |
| HER2 | Native | (K)GLQSLPTHDPSPLQR | 1644.86 | PSPLQR | 697.80 |
| | Calibrant 1 | (K)GLQSLPTDPHSPLQR | 1644.86 | PHSPLQR | 835.45 |
| | Calibrant 2 | (K)GLQSLPSHDPTPLQR | 1644.86 | PTPLQR | 711.83 |

One of skill in the art would, of course, understand that the approach illustrated above also applies to quantification of phosphatases, or a combination of kinases and phosphatases.

5.5.2 Isobaric Mass Tags

Protein kinases and phosphatases from different samples can be quantified using a mass tagging approach. In one embodiment, the methods of the invention include covalently coupling an isobaric mass tag to proteins (e.g., kinases and/or phosphatases) bound to a protein capture agent. Each isobaric mass tag in a set has the same mass as every other mass tag in the set, but a scissile bond in a different position than any other mass tag in the set. In one embodiment, isobaric mass tags comprise a peptide, e.g. those described in U.S. Ser. No. 11/344,801, filed Feb. 1, 2006, incorporated by reference herein in its entirety. In another embodiment, isobaric mass tags are non-peptide mass tags, e.g., those described in U.S. Pat. Application No. 60/860,041, filed Nov. 20, 2006, incorporated by reference herein in its entirety. The captured proteins labeled with the mass tags can be used in methods as described above and/or in examples.

### 6. Kits

The invention also relates to kits for capturing proteins, for example kinases and/or phosphatases. An exemplary kit comprises a capture agent labeled with a first member of an affinity pair, a solid support coated with a second member of the affinity pair, and a set of instructions for use. The capture agent can be any capture agent described above. Likewise, the affinity pair can be any affinity pair described above. In one embodiment, the solid support comprises a multi-well plate coated with the second member of the affinity pair. In some embodiments, the kit also comprises a calibrator peptide and/or a set of isobaric mass tags, as discussed above. Optionally, the kit also comprises one or more proteins or peptides labeled with one or more mass tags, which can be used, for example, for reference or calibration purposes.

### 7. EXAMPLES

### Example 1

### Analysis of HER2 Levels in a Human, Caucasian, Breast, Adenocarcinoma Cell Line (SK-BR-3)

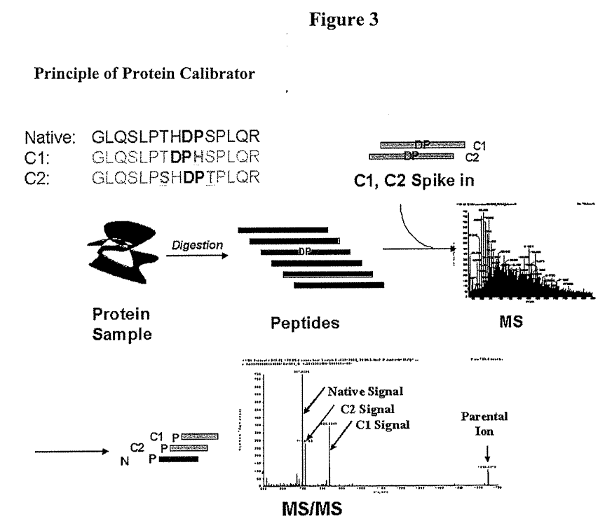

The native and calibrator peptides employed in the Her2 quantification experiment are presented in Table 2. The overall workflow of this experiment is presented in FIG. 3. Once SK-BR-3 cells were grown to 90% confluent, they were washed with ice cold phosphate-buffered saline and lysed to generate whole cell extracts, using 20 mM Hepes buffer (pH 7.9) containing 0.5% (v/v) Nonidet P-40 detergent, 15% (v/v) glycerol, 300 mM NaCl, 1 mM EDTA, 1 mM dithiothreitol, 1 mM sodium vanadate, 10 mM sodium fluoride, 0.5 mM phenylmethylsulfonyl fluoride, and leupeptin, pepstatin, and aprotinin (1 μg/ml each). Cell lysates were incubated on ice for one hour and whole cell extracts were collected by centrifugation for 20 minutes. A total 500 μg of cell lysate was solubilized in denaturing buffer (0.5% SDS, 1 mM TCEP) and heated at 95° C. for 20 minutes. Denatured lysate was clarified using Microcon Centrifugal Filter Devices, 50 kD MWCO (Millipore Corporation, Bedford, Mass.), to eliminate salt and small proteins. The filter-retained proteins were eluted using 75 μl trypsin digestion buffer (50 mM NH₄HCO₃, pH 8.0, 5% acetonitrile). The concentration of the total proteins was 0.33 μg/μl. The proteins were then digested with sequencing grade trypsin at 1:20 (w/w) trypsin-to-protein ratio overnight at 37° C. (100 μl). Peptides were analyzed on a MALDI qTOF mass spectrometer. The samples were spotted on 20×20 MALDI plate (Applied Biosystems) with 0.4 μl/well. The Her2 calibrators were spiked into the tryptic digestion reaction before dilution and the final amount on each well of calibrator 1 and calibrator 2 was 2 femtomoles and 1 femtomole, respectively. In order to quantify Her-2 in the sample, the peak with 1,644.86 dalton mass (parental ion) is selected in the first stage of the mass spectrometer and the resulting fragmented native peptide peak at 697.80 daltons (native signal), obtained in the second stage of the mass spectrometer due to fragmentation of the labile DP bond is compared directly with the known quantities of calibrant peptide peaks, simultaneously resolved in the window, having masses of 835.45 (C1 signal) and 711.83 (C2 signal).

In the cited example, Her2 kinase was enriched only modestly in the SK-BR-3 cell lysate by conventional biochemical methods. In general, those purification methods that deliver substantial enrichment of target kinases require a combination of ammonium sulfate precipitation, ion-exchange chromatography, gel filtration, hydrophobic interaction chromatography and/or dye-ligand chromatography. The specific procedures typically differ from protein kinase to protein kinase and attempt to exploit unique or unusual structural features contained within the enzyme of interest. The classical approaches are not amenable to large-scale enrichment of the entire kinome. Replacement of these classical, multi-step, low-yield, protein purification methods with efficient affinity techniques is crucial to the kinome-wide analyses described herein.

Using the kinase enrichment methods described herein in combination with the tandem mass spectrometry-based absolute quantification strategy provides a first stage MS profile that is significantly simplified (minimizing the need for extensive pre-fractionation by high performance liquid chromatography). The sensitivity of detection of a particular kinase is improved, and a multiplexed kinase analysis is feasible due to the broad spectrum of kinases enriched in the single step. Quantification of tens to hundreds of protein or lipid kinases is a simple matter of spiking the kinase enriched sample with the selected calibrant peptides.

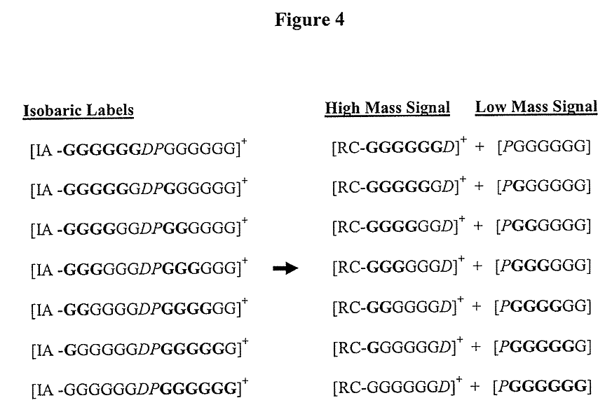

Methods for using a tandem mass spectrometer to simultaneously identify and quantify changes in protein content from multiple complex samples have been described. For example, U.S. Pat. No. 6,824,981 describes use of isobaric mass tags for quantifying protein molecules. These labels are typically isobaric peptides possessing a common amino acid composition, with a cleavage enhancement moiety, which is an aspartic acid (Asp, D) and proline (Pro, P) scissile bond group (see FIG. 4). Distributed around the DP sequence are six isotopically light glycine residues (¹²C₂H₃¹⁴NO) and 6 isotopically heavy glycine residues (¹³C₂H₃¹⁵NO). The amino terminal end of the isobaric peptide tag possesses a reactive group, such as a haloacetyl group, that reacts with the sulfhydryl group in cysteine residues of a protein. Typically, the isobaric labels are conjugated to reduced and denatured intact protein molecules. After trypsin digestion, and during mass analysis, labeled target peptides can ionize and be filtered from other molecules based on mass-to-charge ratio (m/z) in the second stage of the tandem mass spectrometer. The DP scissile bond is generally fragmented under collision-induced dissociation (CID), which gives rise to two quantifiable groups of signals, low mass signals containing label sequences from the proline residue to the C-terminal glycine residue and high mass signals consisting of the target peptide with the label sequence from the N-terminal glycine residue to the aspartate residue. Using the isobaric mass tags, signal to noise ratios are dramatically enhanced because only labeled analytes are selected for CID alteration in tandem mass spectrometry for quantification. Since all labels are isobaric forms of one another, the overall masses of labeled proteins or peptides are always the same. Unlike most isotope tags, these label-conjugated proteins or peptides co-elute in chromatographic separations, providing more accurate quantification. In addition, the two sets of signals (low and high mass signals) can be used in quantification separately or in combination to generate correlating ratios, making quantification more precise.

During use of the multiplexed protein quantification approach described above in reference to FIG. 4, samples to be analyzed are diluted due to combination of the various samples before analysis. Thus, in a seven-plex relative quantification experiment, proteins in the individual samples are diluted seven-fold, and in a 34-plex analysis, achieved by altering the position of the DP dipeptide relative to the heavy and light glycine residues in the isobaric peptide, the dilution factor is 34-fold. Consequently, measurement sensitivity declines substantially in this type of multiplexing experiment, resulting in only the most abundant proteins being amenable to profiling in a given specimen.

The methods described herein for enriching a kinase population, such as a kinome, and quantitating peptides corresponding to members of the kinase population in reference to isobaric reference peptides can provide the sensitivity needed for kinase expression levels to be quantified as a function of a variety of biological phenomenon, including pharmacological treatment with a drug, exposure to a toxicological compound or hormone-induced differentiation of a cell line.

For example, protein specimens, representing seven different physiological or pathological states under investigation, are prepared in 0.05% Tween-20 detergent and 1 mM DTT in phosphate-buffered saline, centrifuged at 6,000×g and filtered (0.2 μm), in order to remove cellular debris. Then, the seven clarified cellular lysates are incubated in seven different wells of a streptavidin-coated 96-well plate that has staurosporine-biotin microcystin-biotin affixed to them. The plates are subsequently washed several times to remove adventitial proteins that do not associate with staurosporine. The captured protein kinases and protein phosphatases in each well are then reduced in 2 mM Tris (2-carboxyethyl) phosphine hydrochloride (TCEP) for 15 minutes by heating at 100° C. After cooling, seven isobaric mass tags containing N-terminal iodoacetate groups, shown schematically in FIG. 4, are added to each of the seven samples at a molar ratio of label to protein cysteine residues of roughly ten to one. The reactions are carried out in the dark overnight at room temperature. Then, the seven reaction mixtures are treated with sequencing grade trypsin to elute them from the wells. The resulting peptide samples are then combined and can be desalted and concentrated using reverse-phase C18 tips (Millipore Corp., Bedford, Mass.), before analysis by tandem mass spectrometry analysis. The reduction of the cellular lysate to an enriched kinase population obviates the need for an intervening peptide separation procedure, such as liquid chromatography, prior to tandem mass spectrometry. Analysis can be performed, for example, on a Thermo-Finnigan LTQ ion trap mass spectrometer operating in data dependant mode. The most intense ions are sequentially analyzed by the tandem mass spectrometry. The normalized collision energy setting is typically 35 and a full MS target value of 3×10⁴ as well as a msn target value of 1×10⁴ can be used for the analysis. All other parameters for data dependant analysis can be based upon factory settings provided with the Xcalibur™ version 1.4 software (Thermo Electron). Xcalibur software is a flexible Microsoft Windows-based data system that provides instrument control and data analysis for the entire family of Thermo Electron mass spectrometers and related instruments. Optionally, exogenously added internal peptide calibrants can be employed to provide absolute quantification of the kinases and phosphatases profiled by the relative quantification method. In this instance, synthetic peptides representing select tryptic fragments containing cysteine residues are made and reacted with a cysteine-reactive isobaric peptide. The label can be similar to those shown in FIG. 4, except, for example, the DP bond can be displaced so as there are five glycine residues N-terminal to the DP bond and seven glycine residues are to the C-terminal of the DP bond. The purified and quantified synthetic peptide is then added to the tryptic digest generated from the kinases and phosphatases being analyzed. The synthetic peptide thus generated is readily distinguished from the labeled peptide fragments arising from the biological specimen because the mass of the synthetic peptide will be displaced by the extra glycine residue in the light fragment and the missing glycine residue in the heavy fragment. While relative quantification of protein kinases and protein phosphatases has been illustrated with the peptide isobaric tags described in U.S. Pat. No. 6,824,981, similar workflows are feasible using a variety of other mass tagging strategies, including iTRAQ labels (Applied Biosystems), ICAT labels (Applied Biosystems), SILAC labels (Invitrogen) and the variety of home-brew isotopic labeling approaches available.

The present invention is not to be limited in scope by the specific embodiments disclosed in the examples, which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

Equivalents: Those skilled in the art will recognize, or be able to ascertain, using no more than routine experimentation, numerous equivalents to the specific embodiments described specifically herein. Such equivalents are intended to be encompassed in the scope of the following claims.

A number of references have been cited, the entire disclosures of which have been incorporated herein in their entirety.

What is claimed is:

1. A method for analysis of proteins in a sample comprising:
a) contacting the sample with a first protein capture agent;
b) separating the proteins bound to the first protein capture agent from the sample;
c) digesting the proteins bound to the first protein capture agent with a protease to provide protein fragments having a scissile bond; and
d) analyzing the products of the protease digestion by mass spectrometry.

2. The method of claim 1, wherein the first protein capture agent is a kinase capture agent.

3. The method of claim 2, wherein the kinase capture agent is a non-selective kinase capture agent.

4. The method of claim 1, wherein the kinase capture agent is a kinase inhibitor.

5. The method of claim 4, wherein the kinase inhibitor is staurosporine or a staurosporine analog.

6. The method of claim 5, wherein the staurosporine analog is selected from the group consisting of 7-hydroxystaurosporine, N-benzoylstaurosporine, 3-hydroxy-4′-N-methylstaurosporine, 3-hydroxy-4′-N-demethylstaurosporine, 3′-demethoxy-3′-hydroxy-4′-N-demethylstaurosporine, staurosporine aglycone and 4′-N-benzoyl staurosporine.

7. The method of claim 4, wherein the kinase inhibitor is selected from the group consisting of KT 5720, K252, H-9, rottlerin, quercetin, hymenialdisine, SB 203580, myricetin, SU11248, roscovitine, EKB569 and SB202190.

8. The method of claim 1, wherein the first protein capture agent is labeled with a first member of an affinity pair.

9. The method of claim 8, wherein the first member of an affinity pair is biotin.

10. The method of claim 1, wherein the first protein capture agent is a phosphatase capture agent.

11. The method of claim 10, wherein the phosphatase capture agent is a phosphatase inhibitor.

12. The method of claim 11, wherein the phosphatase inhibitor is selected from the group consisting of okadaic acid, tautomycin, microcystin, a microcystin derivative, calyculin A, calyculin B, calyculin C, calyculin D, calyculin E, calyculin F, calyculin G, calyculin H, cantharidin, thyrsferyl 23-acetate, isopalinurin, dragacidin, a dragacidin derivative, fostriecin, 1-(oxalyl-amino)-4,5,6,7-tetrahydro-thieno[2,3-c]pyridine-3-carboxylic acid and bis-(maltoato)-oxovanadium(IV).

13. The method of claim 10, wherein the phosphatase capture agent is labeled with a first member of an affinity pair.

14. The method of claim 13, wherein the first member of an affinity pair is biotin.

15. The method of claim 1, wherein the protease is trypsin.

16. The method of claim 1, wherein mass spectroscopy is tandem mass spectroscopy.

17. The method of claim 1, further comprising
a) contacting the sample with a second protein capture agent;
b) separating the proteins bound to the second protein capture agent from the sample; and
c) digesting the proteins bound to the second protein capture agent with a protease to provide protein fragments comprising a scissile bond.

18. The method of claim 17, wherein the first protein capture gent is a kinase capture agent and the second protein capture agent is a kinase capture agent different from the first kinase capture agent.

19. The method of claim 17, wherein the first protein capture agent is a kinase capture agent and the second protein capture agent is a phosphatase capture agent.

20. The method of claim 1, further comprising:
a) providing to protein fragments having a scissile bond a calibrator peptide having a scissile bond and having the same amino acid composition and same mass as a protein fragment after protease digestion, wherein the calibrator peptide has a scissile bond in a different location from the protein fragment; and
b) analyzing the calibrator peptide by mass spectroscopy.

21. The method of claim 20, wherein mass spectroscopy is tandem mass spectroscopy.

22. A method for analysis of proteins from a plurality of samples comprising:
a) contacting each sample with a protein capture agent;
b) separating the proteins bound to the protein capture agent from each sample;
c) coupling a set of isobaric mass tags to the captured proteins or protein fragments, wherein proteins in each sample are coupled with a different isobaric mass tag from the set and wherein each isobaric mass tag in the set has a scissile bond in a different position than any other mass tag in the set;
d) digesting the captured proteins with a protease to provide protein fragments; and
e) detecting a plurality of isobaric mass tags by mass spectrometry in the same experiment.

23. The method of claim 22, wherein the isobaric mass tags are coupled to the captured proteins prior to digestion with a protease.

24. The method of claim 22, wherein the isobaric mass tags are coupled to the protein fragments resultant from the digestion of captured proteins with a protease.

25. The method of claim 22, wherein each isobaric mass tag comprises a peptide.

26. The method of claim 22, wherein the scissile bond is Asp-Pro bond.

27. The method of clam 22, wherein mass spectrometry is tandem mass spectrometry.

28. The method of claim 22, wherein the first protein capture agent is a kinase capture agent.

29. The method of claim 28, wherein the kinase protein capture agent is a non-selective kinase capture agent.

30. The method of claim 28, wherein the kinase capture agent is a kinase inhibitor.

31. The method of claim 30, wherein the kinase inhibitor is staurosporine or a staurosporine analog.

32. The method of claim 31, wherein the staurosporine analog is selected from the group consisting of 7-hydroxystaurosporine, N-benzoylstaurosporine, 3-hydroxy-4′-N-methylstaurosporine, 3-hydroxy-4′-N-demethylstaurosporine, 3′-demethoxy-3′-hydroxy-4′-N-demethylstaurosporine, staurosporine aglycone and 4′-N-benzoyl staurosporine.

33. The method of claim 30, wherein the kinase inhibitor is selected from the group consisting of KT 5720, K252, H-9, rottlerin, quercetin, hymenialdisine, SB 203580, myricetin, SU11248, roscovitine, EKB569 and SB202190.

34. The method of claim 22, wherein the first protein capture agent is labeled with a first member of an affinity pair.

35. The method of claim 34, wherein the first member of an affinity pair is biotin.

36. The method of claim 22, wherein the first protein capture agent is a phosphatase capture agent.

37. The method of claim 36, wherein the phosphatase capture agent is a phosphatase inhibitor.

38. The method of claim 37, wherein the phosphatase inhibitor is selected from the group consisting of okadaic acid, tautomycin, microcystin, a microcystin derivative, calyculin A, calyculin B, calyculin C, calyculin D, calyculin E, calyculin F, calyculin G, calyculin H, cantharidin, thyrsferyl 23-acetate, isopalinurin, dragacidin, a dragacidin derivative, fostriecin, 1-(oxalyl-amino)-4,5,6,7-tetrahydro-thieno[2,3-c]pyridine-3-carboxylic acid and bis-(maltoato)-oxovanadium(IV).

39. The method of claim 22, wherein the protease is trypsin.

40. The method of claim 22, wherein mass spectroscopy is tandem mass spectroscopy.

41. The method of claim 40, further comprising:
a) providing to the protein fragments a calibrator peptide having a scissile bond and having the same amino acid composition and same mass as each isobaric mass tag in the set, wherein the calibrator peptide has a scissile bond in a different location from every isobaric mass tag in the set;
b) detecting the calibrator peptide by mass spectrometry; and
c) quantitatively correlating the mass spectrometry signals from the mass tag with the mass spectrometry signals from the calibrator peptide.

42. A method for isolating a plurality of proteins from a sample comprising:
a) providing a first kinase capture agent and a second protein capture agent;
b) contacting the sample with the first kinase capture agent and the second protein capture agent;
c) separating the proteins bound to the first kinase capture agent and the second protein capture agent from the sample.

43. The method of claim 42, wherein the first kinase capture agent is a non-selective kinase capture agent.

44. The method of claim 42, wherein the first kinase capture agent is a kinase inhibitor.

45. The method of claim 43, wherein the kinase inhibitor is staurosporine or a staurosporine analog.

46. The method of claim 45, wherein the staurosporine analog is selected from the group consisting of 7-hydroxystaurosporine, N-benzoylstaurosporine, 3-hydroxy-4′-N-methylstaurosporine, 3-hydroxy-4′-N-demethylstaurosporine, 3′-demethoxy-3′-hydroxy-4′-N-demethylstaurosporine, staurosporine aglycone and 4′-N-benzoyl staurosporine.

47. The method of claim 42, wherein the first kinase capture agent is labeled with a first member of an affinity pair.

48. The method of claim 47, wherein the first member of an affinity pair is biotin.

49. The method of claim 42, wherein the second protein capture agent is a second kinase capture agent different from the first kinase capture agent.

50. The method of claim 49, wherein the second kinase capture agent is a kinase inhibitor.

51. The method of claim 50, wherein the second kinase inhibitor is selected from the group consisting of KT 5720, K252, H-9, rottlerin, quercetin, hymenialdisine, SB 203580, myricetin, SU11248, roscovitine, EKB569 and SB202190.

52. The method of claim 49, wherein the second kinase capture agent is labeled with a first member of an affinity pair.

53. The method of claim 52, wherein the first member of an affinity pair is biotin.

54. The method of claim 42, wherein the second protein capture agent is a phosphatase capture agent.

55. The method of claim 54, wherein the phosphatase capture agent is a phosphatase inhibitor.

56. The method of claim 55, wherein the phosphatase inhibitor is selected from the group consisting of okadaic acid, tautomycin, microcystin, a microcystin derivative, calyculin A, calyculin B, calyculin C, calyculin D, calyculin E, calyculin F, calyculin G, calyculin H, cantharidin, thyrsferyl 23-acetate, isopalinurin, dragacidin, a dragacidin derivative, fostriecin, 1-(oxalyl-amino)-4,5,6,7-tetrahydro-thieno[2,3-c]pyridine-3-carboxylic acid and bis-(maltoato)-oxovanadium(IV).

57. The method of claim 54, wherein the phosphatase capture agent is labeled with a first member of an affinity pair.

58. The method of claim 57, wherein the first member of an affinity pair is biotin.

59. A kit comprising:
a) a capture agent labeled with a first member of an affinity pair;
b) a plate having one or more wells, wherein each well is coated with a second member of the affinity pair; and
c) a set of instructions for use.

60. The kit of claim 59, wherein the plate has 2, 4, 8, 16, 64, 96, 128, 256, 384 or 512 wells.

61. The kit of claim 60, further comprising a set of calibrator peptides.

62. The kit of claim 60, further comprising a set of isobaric mass tags.

63. The kit of claim 60, wherein the first member of the affinity pair is biotin.

64. The kit of claim 63, wherein the second member of the affinity pair streptavidin.

65. The kit of claim 60, wherein the capture agent is a kinase capture agent.

66. The kit of claim 60, wherein the kinase capture agent is a non-selective kinase capture agent.

67. The kit of claim 65, wherein the kinase capture agent is a kinase inhibitor.

68. The kit of claim 67, wherein the kinase inhibitor is staurosporine or a staurosporine analog.

69. The kit of claim 68, wherein the staurosporine analog is selected from the group consisting of 7-hydroxystaurosporine, N-benzoylstaurosporine, 3-hydroxy-4′-N-methylstaurosporine, 3-hydroxy-4′-N-demethylstaurosporine, 3′-demethoxy-3′-hydroxy-4′-N-demethylstaurosporine, staurosporine aglycone and 4′-N-benzoyl staurosporine.

70. The kit of claim 67, wherein the kinase inhibitor is selected from the group consisting of KT 5720, K252, H-9, rottlerin, quercetin, hymenialdisine, SB 203580, myricetin, SU11248, roscovitine, EKB569 and SB202190.

71. The kit of claim 60, wherein the capture agent is a phosphatase capture agent.

72. The kit of claim 71, wherein the phosphatase capture agent is a phosphatase inhibitor.

73. The kit of claim 72, wherein the phosphatase inhibitor is selected from the group consisting of okadaic acid, tautomycin, microcystin, a microcystin derivative, calyculin A, calyculin B, calyculin C, calyculin D, calyculin E, calyculin F, calyculin G, calyculin H, cantharidin, thyrsferyl 23-acetate, isopalinurin, dragacidin, a dragacidin derivative, fostriecin, 1-(oxalyl-amino)-4,5,6,7-tetrahydro-thieno[2,3-c]pyridine-3-carboxylic acid and bis-(maltoato)-oxovanadium(IV).
